(19) United States
(12) Patent Application Publication
(10) Pub. No.: US 2005/0004215 A1
(43) Pub. Date: Jan. 6, 2005

(54) Amorphous simvastatin calcium and methods for the preparation thereof

(76) Inventors: Ferenc Korodi, Debrecen (HU); Erika Feher, Debrecen (HU); Csaba Szabo, Debrecen (HU); Istvan Bodi, Balmazujvaros (HU); Adrienne Kovacsne-Mezei, Debrecen (HU)

Correspondence Address: KENYON & KENYON, One Broadway, New York, NY, 10004-1050, US

(21) Appl. No.: 10/815,362

(22) Filed: Apr. 1, 2004

Related U.S. Application Data
(60) Provisional application No. 60/459,352, filed on Apr. 1, 2003.

(51) Int. Cl. C07C 69/66; A61K 31/225

(52) U.S. Cl. 514/548; 560/185

(57) ABSTRACT

An amorphous dihydroxy open acid simvastatin calcium and methods for preparing amorphous simvastatin calcium.

### CROSS-REFERENCE TO RELATED APPLICATION

This application claims the benefit of the U.S. Provisional Application Ser. No. 60/459,352 filed Apr. 1, 2003, the disclosure of which is incorporated by reference in its entirety herein.

### FIELD OF THE INVENTION

The invention relates to amorphous simvastatin calcium and methods for obtaining amorphous simvastatin calcium.

### BACKGROUND OF THE INVENTION

Simvastatin is a synthetic analog of lovastatin, wherein the 8-acyl moiety is 2,2-dimethylbutyryl. Simvastatin is chemically designated as 2,2-dimethylbutanoic acid (4R,6R)-6-[2[1S,2S, 6R,8S,8aR)-1,2,6,7,8,8a-hexahydro-2,6-dimethyl-1-[2-(tetrahydro-4-hydroxy-6-oxo-2H-pyran-2-yl)ethyl]-1-napthalenyl ester (CAS Registry No. 79902-63-9). The chemical structure of simvastatin is:

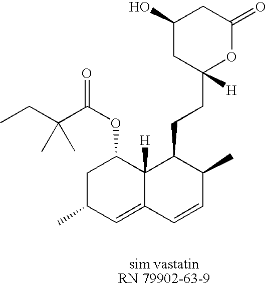

Simvastatin is now commercially available as ZOCOR® in some markets. The preparation of simvastatin was originally described in U.S. Pat. No. 4,444,784. The process involves deacylation of lovastatin followed by a subsequent acylation with the 2,2-dimethylbutyryl moiety. Simvastatin is also prepared by the alpha alkylation of the lovastatin ester moiety as described in U.S. Pat. Nos. 4,582,915 and 4,820,850.

Both simvastatin and lovastatin are members of the statin family and are potent anti-hypercholesterolemic agents. They both inhibit the enzyme 3-hydroxy-3-methyl-glutarylcoenzyme A reductase (“HMG-CoA reductase”) which catalyzes the formation of mevalonic acid, and thus inhibit cholesterol biosynthesis. They also increase the number of cellular LDL-receptors which remove the LDL cholesterol circulating in the blood, and thereby lower blood cholesterol levels. Simvastatin is a more potent HMG-CoA reductase inhibitor as compared to lovastatin.

Both simvastatin and lovastatin can exist either in a 3-hydroxy lactone ring form or a dihydroxy open acid form. The lactonized form is not an active inhibitor of HMG-CoA reductase, but the dihydroxy open acid form is. The intramolecular condensation of the dihydroxy open acid form to the corresponding lactonized form occurs under acidic conditions (e.g., in the stomach where pH is about pH 4 or under). It is desirable to prepare simvastatin in the dihydroxy open acid form to limit the in vivo amount of inactive of lactone.

WO 00/53566 discloses a crystalline calcium salt of dihydroxy open acid simvastatin form and the preparation thereof, particularly a hydrated calcium salts characterized by corresponding x-ray powder diffraction, thermogravimetry, differential scanning calorimetry and solid state ¹³C-NMR spectroscopy data.

WO 00/53566 discloses two synthesis methods for preparing the crystalline dihydroxy open acid simvastatin calcium salt hydrate. The first synthesis method relates to hydrolyzing simvastatin lactone form in an inorganic base e.g., sodium hydroxide and water or in a mixture of water and an organic solvent, and treating the hydrolyzed simvastatin with Ca(OAc)₂.H₂O to form the target salt followed by precipitation of the target. The second synthesis method relates to combining an ammonium salt of dihydroxy open acid simvastatin (as a starting material) with Ca(OAc)₂.H₂O to obtain a crystalline hydrate form of simvastatin calcium salt. Starting with a dihydroxy open simvastatin form avoids the hydrolysis step needed if a lactonized simvastatin is used as a starting material. WO 00/53566 further discloses a delayed-release dosage form of the crystalline hydrated simvastatin calcium salt.

WO 02/20457 discloses the preparation and characterization of five polymorphic crystalline forms of simvastatin calcium salt including both hydrated and anhydrous forms. These different polymorphic crystalline forms are characterized by x-ray powder diffraction, thermogravimetry, differential scanning calorimetry and solid state ¹³C-NMR spectroscopy. WO 02/20457 further discloses methods for making the polymorphic crystalline simvastatin calcium salts forms I, II, III, IV and V. WO 02/20457 discloses form I containing 2.8-3.6 moles of water per mole of calcium and forms II, III, IV and V each having a different degree of hydration achieved by using different drying methods.

There can be many advantages to using the amorphous form of a drug. Two of the most important advantages are enhanced solubility and bioavailability. There is a continuing need to prepare amorphous dihydroxy open acid simvastatin calcium salt.

### SUMMARY OF THE INVENTION

The present invention provides amorphous calcium salt of dihydroxy open acid simvastatin.

The present invention provides amorphous simvastatin calcium. The amorphous form may be characterized by one or more characters selected from the group consisting of a x-ray powder diffraction pattern as shown in FIG. 1, the loss on drying as determined by thermogravimetry weight loss curve (can be 1.5% wt to 2% wt) as shown in FIG. 2, and a differential scanning calorimetry curve as shown in FIG. 3.

According to another aspect, the present invention provides anhydrous amorphous simvastatin calcium containing less than 1.0% wt of water. According to another aspect, the amorphous simvastatin calcium may contain up to about 4% wt of water, typically between about 1.8% and about 2.4% wt of water.

The present invention also provides a process for preparing an amorphous simvastatin calcium, comprising the steps of:
a) combining a salt of dihydroxy open acid simvastatin and a mixture of water and a water-immiscible organic solvent wherein the mixture forms an inorganic phase and an organic phase;
b) adding a calcium containing compound to the mixture; and
c) separating amorphous simvastatin calcium from the organic phase.

Preferably, the simvastatin salt is selected from the group consisting of alkali earth metal salts and ammonium salt. Preferably the alkali earth metal salts is selected from the group consisting of sodium salt or potassium salt.

Preferably, the water-immiscible organic solvent is selected from the group consisting of ether, ester, aromatic hydrocarbon and halogenated hydrocarbon. Preferably the ether has the formula R₁—O—R₂ wherein R₁ is C₁₋₄ alkyl and R₂ is C₁₋₄ alkyl. Preferably the ester has the formula R₁—CO₂—R₂ wherein R₁ is C₁₋₄ alkyl and R₂ is C₁₋₄ alkyl. Preferably the aromatic hydrocarbon is a mono or bicyclic aromatic ring system containing from 6 to 10 carbon atoms which may be optionally substituted by one or two groups selected from C₁₋₄ alkyl, hydroxyl or halogen. Preferably the halogenated hydrocarbon is a C₁₋₄ alkyl group substituted by one to four halogen atoms. Preferably the halogen atoms are chlorine. More preferably, the ether is diethyl ether, the ester is ethyl acetate, the aromatic hydrocarbon is toluene and the halogenated hydrocarbon is dichloromethane.

The calcium containing compound may be either an inorganic or organic calcium salt. Preferably, the calcium salt is selected from the group consisting of calcium chloride, calcium bromide, calcium oxide, calcium hydroxide, calcium acetate and calcium 2-ethyl-hexanoate.

The separating step may be performed by evaporation or precipitation. Preferably, the precipitation is performed by adding an antisolvent selected from the group consisting of acetone, acetonitrile, methanol and hexane. Most preferably, the precipitation is performed by adding acetonitrile.

According to another aspect, the present invention provides a process for preparing an amorphous simvastatin calcium, comprising the steps of:
a) combining a salt of simvastatin with the mixture of water and a water-immiscible organic solvent wherein the mixture forms an inorganic phase and an organic phase;
b) adding an acid to the inorganic phase;
c) separating the organic phase from the inorganic phase;
d) adding a calcium containing compound to the organic phase; and
e) separating amorphous simvastatin calcium from the organic phase.

Preferably, the simvastatin salt is selected from the group consisting of alkali earth metal salts and ammonium salt. Preferably the alkali earth metal salts is selected from the group consisting of sodium salt or potassium salt.

The acid is an inorganic acid or an organic acid. The acid may be selected from the group consisting of hydrobromic acid (HBr), sulfuric acid (H₂SO₄), hydrochloric acid, phosphoric acid (H₃PO₄), propionice and acetic acid. More preferably, the acid is hydrochloric acid.

Preferably, the water-immiscible organic solvent is selected from the group consisting of ether, ester, aromatic hydrocarbon and halogenated hydrocarbon. Preferably the ether has the formula R₁—O—R₂ wherein R₁ is C₁₋₄ alkyl and R₂ is C₁₋₄ alkyl. Preferably the ester has the formula R₁—CO₂—R₂ wherein R₁ is C₁₋₄ alkyl and R₂ is C₁₋₄ alkyl. Preferably the aromatic hydrocarbon is a mono or bicyclic aromatic ring system containing from 6 to 10 carbon atoms which may be optionally substituted by one or two groups selected from C₁₋₄ alkyl, hydroxyl or halogen. Preferably the halogenated hydrocarbon is a C₁₋₄ alkyl group substituted by one to four halogen atoms. Preferably the halogen atoms are chlorine. More preferably, the ether is diethyl ether, the ester is ethyl acetate, the aromatic hydrocarbon is toluene, and the halogenated hydrocarbon is dichloromethane.

Preferably, the calcium containing compound is selected from the group consisting of calcium oxide, calcium hydroxide, or a calcium salt of an organic acid. The organic acid is preferably selected from acetic and 2-ethylhexanoic acid.

The separating step may be performed by evaporation or precipitation. Preferably, the precipitation is performed by adding an antisolvent selected from the group consisting of acetone, acetonitrile, methanol and hexane. Most preferably, the precipitation is performed by adding acetonitrile.

According to another aspect, the present invention provides a process for preparing an amorphous simvastatin calcium, comprising the steps of:
a) combining a simvastatin lactone with a mixture of water and a water miscible organic solvent;
b) hydrolyzing the simvastatin lactone to form a calcium salt of simvastatin; and
c) separating amorphous simvastatin calcium.

Preferably, the water-miscible organic solvent is a good solvent for simvastatin calcium, preferably selected from the group consisting of ethanol and tetrahydrofuran.

Preferably, the hydrolyzing step is performed by calcium hydroxide.

Preferably, the separating step may be performed by evaporation. More preferably, the separating step is performed by precipitation. More preferably, the precipitation is performed by adding an antisolvent selected from the group consisting of acetone, acetonitrile, methanol and water. Most preferably, the precipitation is performed by adding water.

According to another aspect, the present invention provides a process for preparing an amorphous simvastatin calcium, comprising the steps of:
a) providing a slurry of simvastatin lactone in water;
b) hydrolyzing the simvastatin lactone to form a calcium salt of simvastatin; and
c) separating amorphous simvastatin calcium.

Preferably, the separating step is performed by filtration.

Preferably, all of the process steps are performed under nitrogen and/or in the presence of an antioxidant. A preferred antioxidant is butylhydroxytoluene (BHT).

The preferred method for drying amorphous simvastatin calcium is performed in a vacuum oven under nitrogen. More preferably, the drying step is performed at a temperature between about 20° C. to about 50° C.

Preferably, the present invention provides amorphous simvastatin calcium with a purity of at least about 96% to about 99%. Preferably, the total impurity content is less than about 1% by HPLC.

The present invention provides anhydrous amorphous simvastatin calcium containing less than 1.0% wt of water, or amorphous simvastatin calcium containing up to about 4% wt of water, typically between about 1.8% and about 2.4% wt of water.

The present invention provides a pharmaceutical formulation comprising amorphous calcium salt of dihydroxy open acid simvastatin and at least one compound selected from the group consisting of a pharmaceutical carrier and a pharmaceutical diluent.

### BRIEF DESCRIPTION OF THE DRAWINGS

FIG. 1 is a x-ray powder diffraction (XRPD) pattern for an amorphous simvastatin calcium.

FIG. 2 is a thermogravimetry (TG) weight loss curve for an amorphous simvastatin calcium.

FIG. 3 is a differential scanning calorimetry (DSC) curve for an amorphous simvastatin calcium.

### DETAILED DESCRIPTION OF THE INVENTION

“An inhibitor of HMG-CoA reductase” refers to statins which can exists either as a 3-hydroxyl lactone ring or as the corresponding dihydroxy open acid. The term “dihydroxy open acid statins” in its broadest embodiment include amorphous calcium salt of dihydroxy open acid statin or a pharmaceutically acceptable salt thereof. The dihydroxy open acid statin includes lovastatin and simvastatin; preferably, simvastatin.

Unless otherwise specified, % is % wt, both refer to % of wt/wt. % wt of water refers to the weight of water/weight of amorphous simvastatin calcium (including the water).

As used herein:
“antisolvent” refers to a solvent used to induce precipitation for crystallization;
“immiscible” refers to incapacity of forming a mutual solution; e.g., oil and water;
“miscible” refers to a capacity for forming a mutual solution; e.g., water and ethanol;
“crystalline solid” refers to regular crystalline packing in a solid, forming an infinite three-dimensional array, a crystalline solid demonstrating the characteristic crystallinity-diffraction of—X-rays and electrons (e.g., XRPD);
“amorphous” refers to a form of material found in both ionic and molecular systems characterized by solid phases in which there is no long-range order; often, an amorphous solid is in a metastable state and thermodynamics requires that crystallization eventually occur; and
“hydrates” refers to crystals of the drug molecules with different numbers of water molecules.
“slurry” is intended to include stirring particles in a liquid.

The dihydroxy open acid form of the statins is the biologically active form. However, the statins are generally administered to a patient in the lactone form, which is converted to its active metabolite, the hydroxy acid form, in the body. Since only the lactone form is of medical interest, the acid form is converted into the lactone form through a process called lactonization. The process of lactonization is an equilibrium reaction whereby the open dihydroxy acid form is converted into the closed lactone form. Because lactonization is an equilibrium process, to obtain a high yield of the lactone product, some means must be employed to shift the equilibrium to the lactone side of the equation. This equilibrium equation can be depicted as follows:

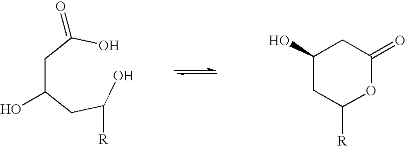

According to one embodiment, the present invention provides an amorphous calcium salt of dihydroxy open acid simvastatin.

Two of the important advantages of the amorphous form are enhanced solubility and bioavailability.

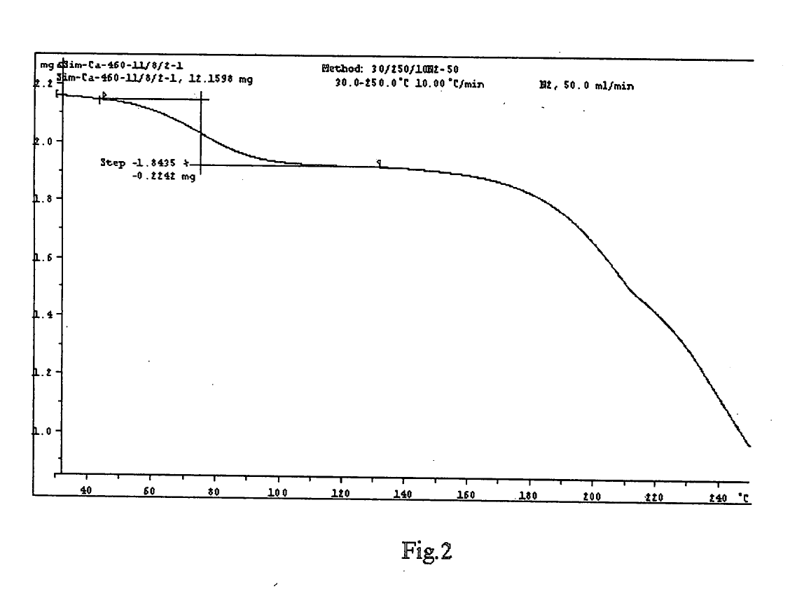

According to another embodiment, the present invention provides an amorphous calcium salt of simvastatin whereby the x-ray powder diffraction pattern (i.e., XRPD) and morphology demonstrate that such calcium salt of simvastatin is amorphous. This particular crystalline hydrated form of simvastatin calcium salt was characterized by X-ray powder diffraction (XRPD), the loss on drying, as determined by thermogravimetry (TGA) can be 1.5% wt to 2% wt, as shown in a typical TGA thermogram of the amorphous form in FIG. 2, and differential scanning calorimetry (DSC).

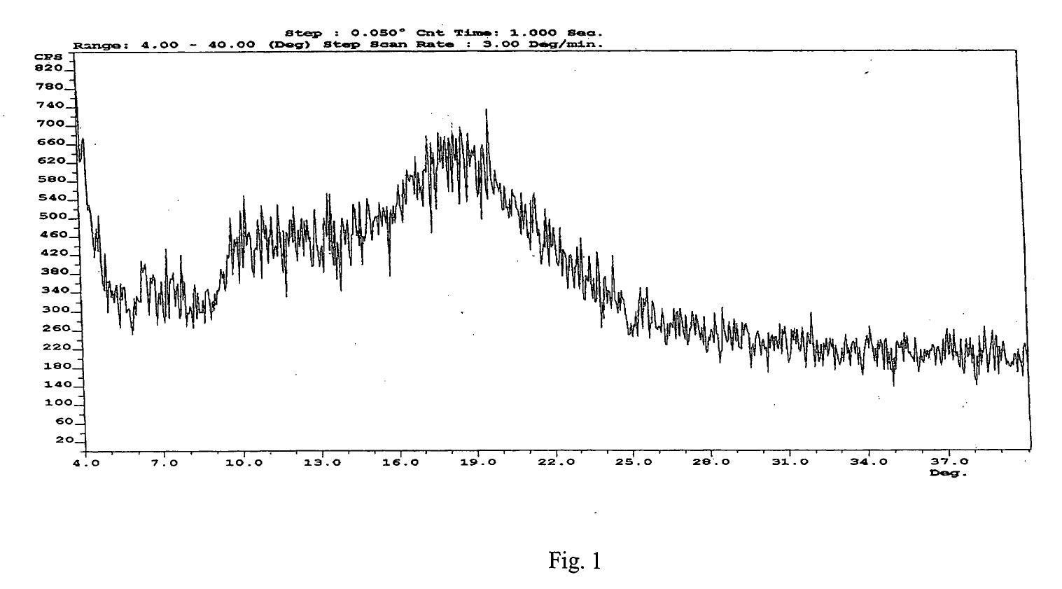

According to another embodiment, the present invention provides an amorphous calcium salt of simvastatin whereby the amorphous form is characterized by a x-ray powder diffraction pattern shown in FIG. 1.

According to another embodiment, the present invention provides an amorphous calcium salt of simvastatin whereby the amorphous form is characterized by a thermogravimetry curve shown in FIG. 2.

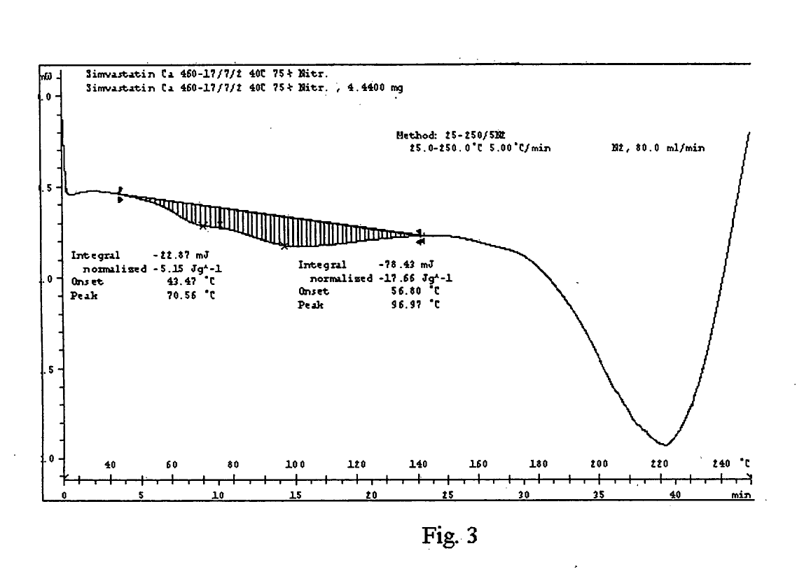

According to another embodiment, the present invention provides an amorphous calcium salt of simvastatin whereby the amorphous form is characterized by a differential scanning calorimetry shown in FIG. 3.

According to another embodiment, the present invention provides an amorphous calcium salt of dihydroxy open acid simvastatin that can be anhydrous or contain water. Preferably, the anhydrous amorphous simvastatin calcium containing less than 1.0% wt of water. According to another aspect, the amorphous simvastatin calcium may contain up to about 4% wt of water, typically between about 1.8% wt and about 2.4% wt of water.

The present invention provides further methods for preparing an amorphous simvastatin calcium.

According to another embodiment, the present invention provides a method for preparation of amorphous simvastatin calcium starting from a salt of simvastatin (preferably, an alkali earth metal or ammonium salt of dihydroxy open acid simvastatin, preferably the alkali earth metal salts is selected from the group consisting of sodium salt or potassium salt) which is combined with a mixture of water and a water-immiscible organic solvent, followed by the addition of the calcium containing compound. Preferably, the water-immiscible organic solvent may be selected from the group consisting of ethers, (e.g., diethyl ether), esters (e.g., ethyl acetate) aromatic hydrocarbons (e.g., toluene) and halogenated hydrocarbons (e.g., dichloromethane). Preferably the ether has the formula R₁—O—R₂ wherein R₁ is C₁₋₄ alkyl and R₂ is C₁₋₄ alkyl. Preferably the ester has the formula R₁—CO₂—R₂ wherein R₁ is C₁₋₄ alkyl and R₂ is C₁₋₄ alkyl. Preferably the aromatic hydrocarbon is a mono or bicyclic aromatic ring system containing from 6 to 10 carbon atoms which may be optionally substituted by one or two groups selected from C₁₋₄ alkyl, hydroxyl or halogen. Preferably the halogenated hydrocarbon is a C₁₋₄ alkyl group substituted by one to four halogen atoms. Preferably the halogen atoms are chlorine. The preferred calcium containing compound may either be an inorganic or organic calcium salt, preferably calcium chloride, calcium bromide, calcium acetate, calcium 2-ethyl-hexanoate, calcium oxide and calcium hydroxide. Phases are separated and amorphous simvastatin calcium is prepared from the organic phase by evaporation or precipitation. Precipitation can be accomplished by addition of an organic solvent which is an antisolvent. The antisolvents include acetone, acetonitrile, methanol, and hexane. Most preferably, acetonitrile is used.

According to another embodiment, the present invention provides a method for preparation of amorphous simvastatin calcium starting from a salt of simvastatin (preferably, an alkali earth metal or ammonium salt of dihydroxy open acid simvastatin, preferably the alkali earth metal salts is selected from the group consisting of sodium salt or potassium salt) which is combined with a mixture of water and a water-immiscible organic solvent. The water-immiscible organic solvent is selected from the group consisting of ethers (e.g., diethyl ether), esters (e.g., ethyl acetate), aromatic hydrocarbons (e.g., toluene) and halogenated hydrocarbons (e.g., dichloromethane) and the like. Preferably the ether has the formula R₁—O—R₂ wherein R₁ is C₁₋₄ alkyl and R₂ is C₁₋₄ alkyl. Preferably the ester has the formula R₁—CO₂—R₂ wherein R₁ is C₁₋₄ alkyl and R₂ is C₁₋₄ alkyl. Preferably the aromatic hydrocarbon is a mono or bicyclic aromatic ring system containing from 6 to 10 carbon atoms which may be optionally substituted by one or two groups selected from C₁₋₄ alkyl, hydroxyl or halogen. Preferably the halogenated hydrocarbon is a C₁₋₄ alkyl group substituted by one to four halogen atoms. Preferably the halogen atoms are chlorine. The water phase is acidified by addition of an inorganic or organic acid. The acid may be selected from the group consisting of hydrobromic acid (HBr), sulfuric acid (H₂SO₄), hydrochloric acid, phosphoric acid (H₃PO₄), propionice and acetic acid, more preferably, hydrochloric acid (HCl). Without being bound by any theory, it is believed that the acid addition forces the salt of dihydroxy open acid simvastatin to enter into the organic phase as dihyroxy open acid simvastatin. Phases are separated and the organic phase containing dihydroxy open acid simvastatin is treated by calcium hydroxide, calcium oxide or a calcium salt of an organic acid (e.g., calcium acetate, calcium 2-ethyl-hexanoate) to form the calcium salt of dihydroxy open acid simvastatin. Amorphous simvastatin calcium salt is prepared by either evaporation or precipitation. Precipitation can be accomplished by addition of an antisolvent exemplified by an organic solvent selected from the group consisting of acetone, acetonitrile, methanol or hexane. The most preferred antisolvent is acetonitrile.

According to yet another embodiment, simvastatin lactone is dissolved in a mixture of water and water miscible organic solvent. Preferably, the water-miscible organic solvent includes ethanol, tetrahydrofuran and the like. Simvastatin lactone is hydrolyzed by calcium hydroxide to form a calcium salt of dihydroxy open acid simvastatin. Amorphous simvastatin calcium is prepared by evaporation or precipitation. Precipitation can be accomplished by addition of an antisolvent. Preferably, the antisolvent includes acetone, acetonitrile, methanol, water and the like. Most preferably, the antisolvent is water.

According to still another embodiment, simvastatin is hydrolyzed by calcium hydroxide as a slurry in water. Amorphous simvastatin calcium is prepared by filtration. Preferably, the process steps are carried out under nitrogen and/or in the presence of an antioxidant. More preferably, the antioxidant is butylhydroxytoluene (BHT). Preferably, the prepared simvastatin calcium product is dried in a vacuum oven under nitrogen at a controlled temperature. More preferably, the temperature is from about 20° C. to about 50° C.

The prepared amorphous simvastatin calcium has a purity of at least about 96% to about 99%. Preferably, the total impurity content is less than about 1% by HPLC.

The prepared amorphous simvastatin calcium may be anhydrous containing less than 1.0% wt of water or contain water up to about 4% wt of water, typically between about 1.8% and about 2.4% wt of water.

The prepared amorphous simvastatin calcium is stable at room temperature during storage in a closed container under nitrogen.

Description of Analytical Methods for the Analysis of Amorphous Simvastatin Calcium

Impurity Content Determination

HPLC method A was used to determine the impurity content of calcium salt of dihydroxy open acid simvastatin. The procedure is summarized as follows:
a) dissolving the sample (i.e., (simvastatin hydroxy acid)₂.Ca salt) in acetonitrile: distilled water (v/v=1:1) diluent;
b) injecting the sample solution (ca. 10 μl) onto a 75.0 mm×4.6 mm, 5 μm RP-18 HPLC column;
c) gradient eluting the column with a mixture of 0.1% phosphoric acid (A) and acetonitrile (B) according to the following profile;
d) measuring the amounts of each impurity at 240 nm wavelength with a UV detector and appropriate recording device;

e) calculating the amount of each impurity referring to simvastatin hydroxy acid ammonium salt working standard at a concentration of 2.0 μg/ml.

HPLC Gradient Profile for HPLC Method A

| Flow rate | Time | Eluent A | Eluent B |
|---|---|---|---|
| [ml/min] | [min] | [v/v %] | [v/v %] |
| 1.5 | 0.0 | 70.0 | 30.0 |
| 1.5 | 8.5 | 54.0 | 46.0 |
| 2.0 | 9.5 | 54.0 | 46.0 |
| 2.0 | 13.0 | 54.0 | 46.0 |
| 2.0 | 22.5 | 15.0 | 85.0 |
| 1.5 | 23.0 | 70.0 | 30.0 |
| 1.5 | 25.0 | 70.0 | 30.0 |

In this method, simvastatin hydroxy acid has a retention time of about 12.8 minutes.
Calcium Salt of Dihydroxy Open Acid Simvastatin Determination

This HPLC method B was used to determine the calcium salt of dihydroxy open acid simvastatin. The procedure is summarized as follows:
a) dissolving the sample ((simvastatin hydroxy acid)₂Ca salt) in acetonitrile:distilled water (1:1) diluent;
b) injecting the sample solution (ca. 10 μl) onto a 75.0 mm×4.6 mm, 5 μm RP-18 HPLC column;
c) gradient eluting at 2.0 ml/min with a mixture of 0.1% phosphoric acid (A) and acetonitrile (B) according to the following profile:
d) measuring of the amounts of each impurity at 240 nm wavelength with a UV detector and appropriate recording device.

e) calculating of the assay referring to simvastatin hydroxy acid ammonium salt working standard at a concentration of 200 μg/ml.

HPLC Gradient Profile for HPLC Method B

| Time | Eluent A | Eluent B |
|---|---|---|
| [min] | [v/v %] | [v/v %] |
| 0.0 | 55.0 | 45.0 |
| 12.0 | 55.0 | 45.0 |
| 12.1 | 10.0 | 90.0 |
| 14.9 | 10.0 | 90.0 |
| 15.0 | 55.0 | 45.0 |

In this method, simvastatin hydroxy acid has a retention time of about 6.9 minutes.

Calcium Content Determination

We used complexometric titration with the Cu-ISE method to determine the calcium content determination of the calcium salt of dihydroxy open acid simvastatin. The procedure is summarized as follows:
a) dissolving ((simvastatin hydroxy acid)₂Ca salt) sample in tetrahydrofuran:sodium-borate buffer pH=10 (1:1) mixture;
b) dosing accurately 4.000 ml copper di-ammonium titriplex solution at a concentration of 0.1 mol/l to the sample solution; and
c) titrating with 0.1 mol/l titriplex solution and determining the endpoint.
   Water Content Determination

We used Karl-Fischer titration method to determine the water content of calcium salt of dihydroxy open acid simvastatin. Specifically, the water content of ((simvastatin hydroxy acid)₂Ca salt) was determined by Karl-Fischer titration in a tetrahydrofuran: methanol (1:1) mixture.

X-Ray Powder Diffraction Pattern Determination The x-ray powder diffraction pattern was taken according to the following conditions:

| | |
|---|---|
| Instrument | ARL-X'TRA - 030 powder diffractometer |
| Roentgen tube | Copper anode (wavelength = 1.5406 A) |
| Detektor | ARL Peltier detector |
| Voltage | 45 KV |
| Current | 40 mA |
| Angle range | 2 Theta = 4-40 degree |
| Step size | 0.05 degree |
| Counting time | 1 sec. |
| Step scan rate | 3.00 Deg/min. |

Thermogravimetry Analysis

The thermogravimetry weight loss curve was taken according to the following conditions:

| | |
|---|---|
| Instrument | Mettler Toledo TGA/SDTA 851e |
| Heating interval | 30-250° C. |
| Heating rate | 10° C./min. |
| Atmosphere | N2 (50 ml/min.) |
| Sample holder | Al-oxide pan 150 μl with pierced lid |

Differential Scanning Calorimetry

The differential scanning calorimetry curve was obtained according to the following conditions:

| | |
|---|---|
| Instrument | Mettler Toledo DSC822e |
| Heating interval | 25-250° C. |
| Heating rate | 5° C./min. |
| Atmosphere | Nitrogen (80 ml/min) |
| Sample holder | Al pan 40 μl with pierced lid |

Other embodiments of the present invention will be more fully understood from the following examples. These examples are intended for illustration purposes of the present invention, but do not in any way limit the scope of the invention.

### EXAMPLES

### Example 1

Simvastatin ammonium salt (11.3 grams, 0.025 mol) was suspended in a mixture of diethyl ether (150 cm³) and water (100 cm³). Aqueous hydrochloric acid (11 cm³, 10% solution) was added to the mixture to adjust the acidity (i.e., pH) of the water phase to between pH 4 and pH 5. The mixture was stirred at room temperature under nitrogen for 10 minutes and the two phases were separated. Calcium hydroxide (0.93 gram, 0.0125 mol) was added to the organic phase containing simvastatin hydroxy acid. The mixture was stirred for 30 minutes and the solution was evaporated to dryness on a rotary evaporator at 45° C. to yield amorphous simvastatin calcium.

Yield: 11.37 grams (100%); assay: 96.1%.

### Example 2

Simvastatin lactone (83.6 grams, 0.20 mol) was dissolved in a mixture of ethanol (1,200 cm³) and water (120 cm³). Calcium hydroxide (14.8 grams, 0.2 mol) was added to the solution and the mixture was stirred at reflux temperature under nitrogen for 1 hour. The reaction mixture was filtered while hot to remove excess calcium hydroxide. Water (1,200 cm³) was added to the filtrate to precipitate the product. The resulting slurry was cooled to 0 to 5° C. and was stirred at this temperature for 2 hours. The precipitate was collected, washed with water and dried in a vacuum oven at 45° C. for 24 hours to yield amorphous simvastatin calcium.

Yield: 56.4 grams (62%); assay: 98.3%; calcium content: 4.2%; water content: 2.3%.

### Example 3

Simvastatin lactone (83.6 grams, 0.2 mol) was dissolved in a mixture of tetrahydrofuran (1,200 cm³) and water (120 cm³). Calcium hydroxide (14.8 grams, 0.2 mol) was added to the solution and the mixture was stirred at room temperature under nitrogen for 1 hour. The reaction mixture was filtrated to remove excess calcium hydroxide. The filtrate was evaporated to dryness on a rotary evaporator. The solid residue was ground in a mortar and dried at 45° C. in a vacuum oven for 24 hours to yield amorphous simvastatin calcium.

Yield: 84.7 grams (93%); assay: 99.6%; calcium content: 4.2%; water content: 1.8%.

### Example 4

Simvastatin ammonium salt (11.3 grams, 0.025 mol) was suspended in a mixture of water (100 cm³) and ethylacetate (150 cm³). Calcium chloride (1.52 grams, 0.0137 mol) was added to the mixture which was then stirred for 0.5 hour. The two phases (i.e., inorganic phase and organic phase) were separated from each other. The organic phase was evaporated to dryness on a rotary evaporator. The solid residue was ground in a mortar and dried at 45° C. in a vacuum oven for 24 hours to yield amorphous simvastatin calcium.

Yield: 10.7 grams (94%); assay: 96.2%.

### Example 5

Simvastatin ammonium salt (11.3 grams, 0.025 mol) was suspended in a mixture of water (100 cm³) and ethylacetate (150 cm³). Calcium hydroxide (1.02 grams, 0.0138 mol) was added to the mixture which was then stirred for 0.5 hour. The two phases (i.e., inorganic phase and organic phase) were separated. The organic phase was evaporated to dryness on a rotary evaporator. The solid residue was ground in a mortar and dried at 45° C. in a vacuum oven for 24 hours to yield amorphous simvastatin calcium.

Yield: 10.7 grams (94%); assay: 96.9%.

### Example 6

Calcium hydroxide (0.49 grams, 0.006 mol) and BHT (0.01 gram) were suspended in water (74 cm³) and heated to 78-82° C. Simvastatin lactone (5.0 grams, 0.012 mol) was added to the slurry and stirred at this temperature for 11.5 hours. The precipitate was collected, washed with water (20 cm³), acetonitrile (20 cm³) and water (20 cm³). It was dried under nitrogen at room temperature in a vacuum oven for 24 hours to yield amorphous simvastatin calcium.

Yield: 4.97 grams (91.4%); assay: 96.5%.

### Example 7

Simvastatin ammonium salt (160 grams, 0.353 mol) was slurried in a mixture of ethylacetate (1,400 cm³) and water (1,400 cm³). Aqueous hydrochloric acid (147 cm³, 10% solution) was added to the mixture to adjust the acidity of the water phase to between pH 3 and pH 4. The mixture was stirred at room temperature under nitrogen for 10 minutes. The two phases (i.e., organic phase and inorganic phase) were separated. The water phase (i.e., inorganic phase) was extracted again with ethylacetate (700 cm³). Calcium hydroxide (13.1 grams, 0.177 mol) was added to the combined organic phase containing simvastatin hydroxy acid. The reaction mixture was stirred for 1 hour at room temperature then was filtered to remove the excess of calcium hydroxide. Acetonitrile (1,710 cm³) was added to the filtrate at 0-5° C. to precipitate the product. The precipitate was collected, washed with acetonitrile (280 cm³), water (280 cm³) and acetonitrile (280 cm³). The washed precipitate was dried in a vacuum oven at 45° C. for 24 hours to yield amorphous simvastatin calcium.

Yield: 144 grams (89.7%); assay: 97.3%.

Pharmaceutical Composition of Simvastatin

Solid-state chemistry of a crystal cannot predicate whether an organic solvent can incorporate into the crystal. The manner in which solvation of a crystal may occur is also unpredictable. There are no rules exist that allow prediction of whether a compound will exist as solvated forms of an organic solvent.

The discovery of new solvated forms of a pharmaceutically useful compound may provide an opportunity to improve the performance characteristics of a pharmaceutical product. It enlarges the repertoire of materials that a formulation scientist has available for designing, for example, a pharmaceutical dosage form of a drug with a targeted release profile or other desired characteristic. It is clearly advantageous when this repertoire is enlarged by the discovery of new solvated crystalline forms of a useful compound.

The present invention relates to the amorphous form of simvastatin. Different crystal forms of simvastatin may possess different physical properties include, for example, the flowability of the milled solid. Flowability affects the ease with which the material is handled during processing into simvastatin. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important physical property of different forms of simvastatin relate to its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

In addition to the active ingredient(s), simvastatin pharmaceutical compositions of the present invention may contain one or more excipients. Excipients are added to the composition for a variety of purposes.

Diluents increase the bulk of a solid pharmaceutical composition and may make a pharmaceutical dosage form containing the composition easier for the patient and care giver to handle. Diluents for solid compositions include, for example, microcrystalline cellulose (e.g. Avice17), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit7), potassium chloride, powdered cellulose, sodium chloride, sorbitol and talc.

Solid pharmaceutical compositions that are compacted into a dosage form like a tablet may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Kluce17), hydroxypropyl methyl cellulose (e.g. Methoce17), liquid glucose, magnesium aluminum silicate, maltodextrin, methylcellulose, polymethacrylates, povidone (e.g. Kollidon7, Plasdone7), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol7, Primellose7), colloidal silicon dioxide, croscarmellose sodium, crospovidone (e.g. Kollidon7, Polyplasdone7), guar gum, magnesium aluminum silicate, methyl cellulose, microcrystalline cellulose, polacrilin potassium, powdered cellulose, pregelatinized starch, sodium alginate, sodium starch glycolate (e.g. Explotab7) and starch.

Glidants can be added to improve the flow properties of non-compacted solid compositions and improve the accuracy of dosing. Excipients that may function as glidants include colloidal silicon dixoide, magnesium trisilicate, powdered cellulose, starch, talc and tribasic calcium phosphate.

When a dosage form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level.

Selection of excipients and the amounts to use may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the pharmaceutical arts.

The disclosures of the cited publications are incorporated herein in their entireties by reference. It is to be understood, however, that the scope of the present invention is not to be limited to the specific embodiments described above. The invention may be practiced other than as particularly described and still be within the scope of the accompanying claims.

1. An amorphous simvastatin calcium salt of dihydroxy open acid simvastatin.

2. The amorphous simvastatin calcium of claim 1, characterized by data selected from the group consisting of a x-ray powder diffraction pattern as shown in FIG. 1, weight loss of about 1.5% to about 2% wt as determine by thermogravimetry and a differential scanning calorimetry curve as shown in FIG. 3.

3. The amorphous simvastatin calcium of claim 2, wherein the amorphous simvastatin calcium is characterized by a x-ray powder diffraction pattern as shown in FIG. 1.

4. The amorphous simvastatin calcium of claim 2, wherein the amorphous simvastatin calcium is characterized by a weight loss of about 1.5% to about 2% wt as determined by thermogravimetry.

5. The amorphous simvastatin calcium of claim 4, wherein the amorphous simvastatin calcium is characterized by a thermogravimetry weight loss curve as shown in FIG. 2.

6. The amorphous simvastatin calcium of claim 2, wherein the amorphous simvastatin calcium is characterized by a differential scanning calorimetry curve as shown in FIG. 3.

7. The amorphous simvastatin calcium of claim 1, wherein the amorphous simvastatin calcium is anhydrous.

8. The amorphous simvastatin calcium of claim 7, wherein the amorphous simvastatin calcium contains less than 1.0% wt of water.

9. The amorphous simvastatin calcium of claim 1, wherein the amorphous simvastatin calcium contains up to about 4% wt of water.

10. The amorphous simvastatin calcium of claim 9, wherein the amorphous simvastatin calcium contains between about 1.8% and about 2.4% wt of water.

11. A process for preparing an amorphous simvastatin calcium, comprising the steps of:
a) combining a salt of simvastatin with a mixture of water and a water-immiscible organic solvent wherein the mixture forms an inorganic phase and an organic phase;
b) adding a calcium containing compound to the mixture; and
c) separating amorphous simvastatin calcium from the organic phase.

12. The process of claim 11, wherein the salt of simvastatin is selected from the group consisting of an alkali earth metal salt and an ammonium salt.

13. The process of claim 11, wherein the salt of simvastatin is dihydroxy open acid simvastatin salt.

14. The process of claim 12, wherein the alkali earth metal salt is sodium salt or potassium salt.

15. The process of claim 11, wherein water-immiscible organic solvent is selected from the group consisting of ether, ester, aromatic hydrocarbon and halogenated hydrocarbon.

16. The process of claim 15, wherein the ether has the formula R₁—O—R₂, wherein R₁ is C₁₋₄ alkyl and R₂ is C₁₋₄ alkyl.

17. The process of claim 15, wherein the ester has the formula R₁—CO₂—R₂, wherein R₁ is C₁₋₄ alkyl and R₂ is C₁₋₄ alkyl.

18. The process of claim 15, wherein the aromatic hydrocarbon is a mono or bicyclic aromatic ring system containing from 6 to 10 carbon atoms which may be optionally substituted by at lease one compound selected from the group consisting of C₁₋₄ alkyl, hydroxyl and halogen.

19. The process of claim 15, wherein the halogenated hydrocarbon is a C₁₋₄ alkyl group substituted by one to four halogen atoms.

20. The process of claim 19, wherein the halogen atom is chlorine.

21. The process of claim 15, wherein the ether is diethyl ether.

22. The process of claim 15, wherein the ester is ethyl acetate.

23. The process of claim 15, wherein the aromatic hydrocarbon is toluene.

24. The process of claim 15, wherein the halogenated hydrocarbon is dichloromethane.

25. The process of claim 11, wherein the calcium containing compound is a calcium salt of an acid selected from the group consisting of inorganic acid and organic acid.

26. The process of claim 25, wherein the calcium salt of an inorganic acid is selected from the group consisting of calcium chloride and calcium bromide.

27. The process of claim 25, wherein the calcium salt of an organic acid is selected from the group consisting of calcium acetate and calcium 2-ethyl-hexanoate.

28. The process of claim 25, wherein the calcium containing compound is selected from the group containing calcium oxide and calcium hydroxide.

29. The process of claim 11, wherein the separating step is performed by evaporation or precipitation.

30. The process of claim 29, wherein the precipitation is performed by adding an antisolvent selected from the group consisting of acetone, acetonitrile, methanol and hexane.

31. The process of claim 29, wherein the precipitation is performed by adding acetonitrile.

32. A process for preparing an amorphous simvastatin calcium, comprising the steps of:
a) combining a salt of simvastatin with a mixture of water and a water immiscible organic solvent wherein the mixture forms an inorganic phase and an organic phase;
b) adding an acid to the inorganic phase;
c) separating the organic phase from the inorganic phase;
d) adding a calcium containing compound to the organic phase; and
e) separating amorphous simvastatin calcium from the organic phase.

33. The process of claim 32, wherein the salt of simvastatin is the salt of dihydroxy open acid simvastatin.

34. The process of claim 32, wherein the acid is an inorganic acid or an organic acid.

35. The process of claim 34, wherein the inorganic acid is selected from the group consisting of hydrobromic acid, sulfuric acid, hydrochloric acid and phosphoric acid.

36. The process of claim 34, wherein the organic acid is selected from the group consisting of propionice and acetic acid.

37. The process as in one of claims 35 and 36, wherein the inorganic acid is hydrochloric acid.

38. The process of claim 32, wherein the salt of simvastatin is selected from the group consisting of an alkali earth metal salt and an ammonium salt.

39. The process of claim 38, wherein the alkali earth metal salt is sodium salt or potassium salt.

40. The process of claim 32, wherein water-immiscible organic solvent is selected from the group consisting of ether, ester, aromatic hydrocarbon and halogenated hydrocarbon.

41. The process of claim 40, wherein the ether has the formula R₁—O—R₂ wherein R₁ is C₁₋₄ alkyl and R₂ is C₁₋₄ alkyl.

42. The process of claim 40, wherein the ester has the formula R₁—CO₂—R₂ wherein R₁ is C₁₋₄ alkyl and R₂ is C₁₋₄ alkyl.

43. The process of claim 40, wherein the aromatic hydrocarbon is a mono or bicyclic aromatic ring system containing from 6 to 10 carbon atoms which may be optionally substituted by at least one compound selected from the group consisting of C₁₋₄ alkyl, hydroxyl and halogen.

44. The process of claim 40, wherein the halogenated hydrocarbon is a C₁₋₄ alkyl group substituted by one to four halogen atoms.

45. The process of claim 44, wherein the halogen atom is chlorine.

46. The process of claim 40, wherein the ether is diethyl ether.

47. The process of claim 40, wherein the ester is ethyl acetate.

48. The process of claim 40, wherein the aromatic hydrocarbon is toluene.

49. The process of claim 40, wherein the halogenated hydrocarbon is dichloromethane.

50. The process of claim 32, wherein the calcium containing compound is a calcium slat of an acid selected from the group consisting of inorganic acid and organic acid.

51. The process of claim 32, wherein the calcium containing compound is selected from the group consisting of calcium oxide and calcium hydroxide.

52. The process of claim 50, wherein the calcium salt of an organic acid is selected from the group consisting of calcium acetate and calcium 2-ethyl-hexanoate.

53. The process of claim 32, wherein the separating step is performed by evaporation or precipitation.

54. The process of claim 53, wherein the precipitation is performed by adding an antisolvent selected from the group consisting of acetone, acetonitrile, methanol and hexane.

55. The process of claim 53, wherein the precipitation is performed by adding acetonitrile.

56. A process for preparing an amorphous simvastatin calcium, comprising the steps of:
a) combining a simvastatin lactone with a mixture of water and a water-miscible organic solvent;
b) hydrolyzing the simvastatin lactone to form a calcium salt of simvastatin; and
c) separating amorphous simvastatin calcium.

57. The process of claim 56, wherein the water-miscible organic solvent is selected from the group consisting of ethanol and tetrahydrofuran.

58. The process of claim 56, wherein the hydrolyzing step is performed using calcium hydroxide.

59. The process of claim 56, wherein the separating step is performed by evaporation.

60. The process of claim 56, wherein the separating step is performed by precipitation.

61. The process of claim 60, wherein the precipitation is performed by adding an antisolvent selected from the group consisting of acetone, acetonitrile, methanol and water.

62. The process of claim 61, wherein the precipitation is performed by adding water.

63. A process for preparing an amorphous simvastatin calcium, comprising the steps of:
a) providing a slurry of simvastatin lactone in water;
b) hydrolyzing the simvastatin lactone to form a calcium salt of simvastatin; and
c) separating amorphous simvastatin calcium.

64. The process of claim 63, wherein steps a-c) are performed under nitrogen.

65. The process of claim 63, wherein steps a-c) are performed in the presence of an antioxidant.

66. The process of claim 65, wherein the antioxidant is butylhydroxytoluene.

67. The process of claim 63, wherein the separating step is performed by filtration.

68. The process of claim 63, further comprising the step of drying amorphous simvastatin calcium in a vacuum oven under nitrogen.

69. The process of claim 68, wherein the drying step is performed at a temperature between about 20° C. to about 50° C.

70. An amorphous simvastatin calcium produced by the process as in one of claims 11, 32, 56 and 63.

71. The amorphous simvastatin calcium of claim 70, wherein the amorphous simvastatin calcium has a purity of at least about 96% to about 99%.

72. A process for preparing a simvastatin lactone, comprising the step of converting the amorphous simvastatin calcium as in one of claims 1-10, 70 and 71 to simvastatin lactone.

73. The process as in one of claims 11, 32, 56 and 63, further comprising the step of converting the amorphous simvastatin calcium to simvastatin lactone.
